(19) United States
(12) Patent Application Publication
(10) Pub. No.: US 2007/0276022 A1
(43) Pub. Date: Nov. 29, 2007

(54) Optically Active Carboxamides

(76) Inventors: Ralf Dunkel, Lyon (FR); Hans-Ludwig Elbe, Wuppertal (DE); Heiko Rieck, Foy-les-Lyon (DE); Benoit Hartmann, Langenfeld (DE); Jorg Nico Greul, Leichlingen (DE); Ulrike Wachendorff-Neumann, Neuwied (DE); Peter Dahmen, Neuss (DE); Karl-Heinz Kuck, Langenfeld (DE); Anne Suty-Heinze, Langenfeld (DE)

Correspondence Address: STERNE, KESSLER, GOLDSTEIN & FOX P.L.L.C., 1100 NEW YORK AVENUE, N.W., WASHINGTON, DC, 20005, US

(21) Appl. No.: 10/583,312

(22) PCT Filed: Dec. 6, 2004
(86) PCT No.: PCT/EP04/13834

§ 371 Date: Apr. 26, 2007

(30) Foreign Application Priority Data
Dec. 18, 2003 (DE) 103 59 511.2
Jan. 28, 2004 (DE) 10 2004 004141.5
Feb. 4, 2004 (DE) 10 2004 005 317.0

(51) Int. Cl. A01N 37/18; A01N 43/10; A01N 43/36; A01N 43/56; A61K 31/501; C07C 233/00; C07D 207/30; C07D 231/10; C07D 409/00

(52) U.S. Cl. 514/406; 514/422; 514/427; 514/617; 548/365.7; 548/375.1; 548/527; 548/561; 564/184

(57) ABSTRACT

New optically active carboxamides of structure (I) 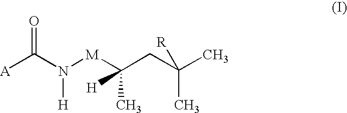 in which R, M and A have the meanings defined in the description, several methods for their preparation of these compounds and their use for the control of detrimental microorganisms as well as new intermediates and their preparation.

The present invention concerns new optically active carboxamides, several methods for their preparation and their use for the control of detrimental microorganisms.

It is already known that numerous carboxamides possess fungicidal properties (c.f. e.g. WO 03/010149, WO 02/059086, WO 02/38542, WO 00/09482, DE-A 102 29 595, EP-A 0 591 699, EP-A 0 589 301 and EP-A 0 545 099). Thus, for example, the racemates of 5-fluoro-1,3-dimethyl-N-[2-(1,3,3-trimethylbutyl)phenyl]-1H-pyrazole-4-carboxamide are known from WO 03/010149 and those of N-[2-(1,3-dimethylbutyl)phenyl]-2-iodobenzamide from DE-A 102 29 595. The activity of these compounds is good, but in many cases leaves much to be desired when they are applied at low concentrations.

Owing to the numerous demands imposed upon modern pest control agents, for example those which affect level of activity, duration of activity, spectrum of activity, range of application, toxicity, combination with other active compounds, combination with formulation excipients or synthesis and owing to the possible appearance of resistance the development of such compounds can never be regarded as concluded. Consequently there is a continuous high demand for new compounds that provide in certain aspects at least partial advantages opposite the known compounds.

New optically active carboxamides of structure (I) have now been found 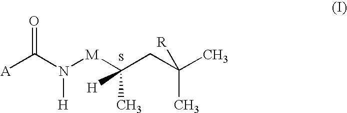
in which

R stands for hydrogen, fluorine, chlorine, methyl, ethyl or trifluoromethyl,

M stands for 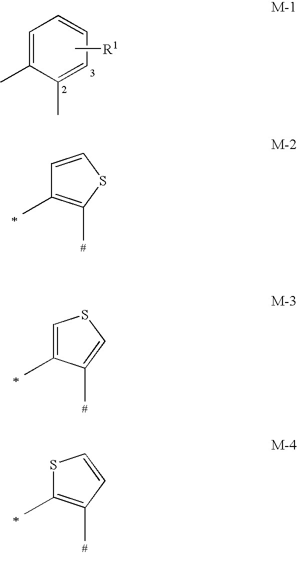
wherein the bonds marked with * is coupled with the amide and the bond marked with # is coupled with the alkyl side chain,

R¹ stands for hydrogen, fluorine, chlorine, methyl or trifluoromethyl,

A stands for the group of structure (A1) 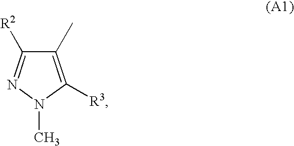
in which
R² stands for methyl, trifluoromethyl or difluoromethyl,
R³ stands for hydrogen, fluorine or chlorine,
   or

A stands for the group of structure (A2) 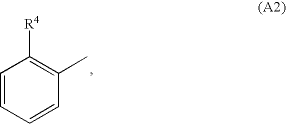
in which
R⁴ stands for trifluoromethyl, chlorine, bromine or iodine,
   or

A stands for the group of structure (A3) 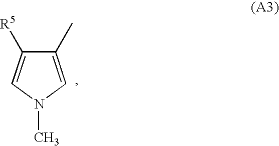
in which
R⁵ stands for methyl, trifluoromethyl or difluoromethyl.

The compounds of structure (I) possess S configuration [C atom labelled with S in structure (I)].

Furthermore it was found that optically active carboxamides of structure (I) are obtained when

a) carboxylic acid derivates of structure (II) 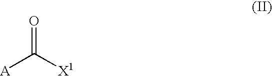
in which
A has the meanings defined above and
X¹ stands for halogen or hydroxy,
are reacted with an amine of structure (III) 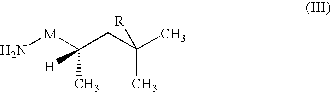
in which R and M have the meanings defined above,
optionally in the presence of a catalyst, optionally in the presence of a condensation agent,
optionally in the presence of an acid binding agent and optionally in the presence of a diluent
   or

b) racemic compounds of structure (I-rac) 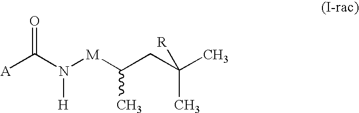
in which R, M and A have the meanings defined above,
are chromatographed on a chiral silica gel stationary phase in the presence of an eluent or eluent mixture as the liquid phase,
or are fractionally crystallised with optically active acids under salt formation and subsequently the enantiomerically pure or enriched compounds of structure (I) is released,
   or

c) compounds of structure (IV) 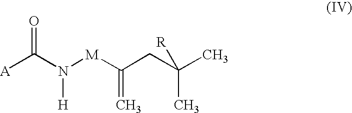
in which R, M and A have the meanings defined above,
or compounds of structure (V) 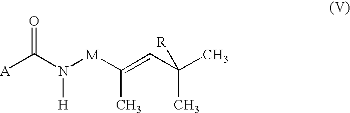
in which R, M and A have the meanings defined above,
or mixtures of both compounds are hydrogenated in the presence of an optically active catalyst or a catalyst with optically active ligand.

Finally it was found that the new optically active carboxamides of structure (I) possess very good microbicidal properties and are suitable for the control detrimental microorganisms both in plant protection and in the protection of materials.

The new optically active carboxamides of structure (I) are characterised opposite known compounds above all by improved action and lower application concentrations and thus lower adverse environmental impact and reduced toxicity.

The optically active carboxamides of the invention are defined in general terms by structure (I). Preferred group definitions of the previously and hereinafter defined structures are given below. These definitions apply in equal measure to the final products of structure (I) as well as for all intermediates.
R stands preferably for hydrogen, methyl or ethyl.
R stands more preferably for hydrogen or methyl.
M stands preferably for M-1.
M stands furthermore preferably for M-2.
M stands furthermore preferably for M-3.
M stands furthermore preferably for M-4.
M stands more preferably for M-1, whereby R¹ stands for hydrogen.
M stands furthermore more preferably for M-2, whereby R¹ stands for hydrogen.
R¹ stands preferably for hydrogen.
R¹ stands furthermore preferably for fluorine, whereby fluorine is more preferably at positions 4, 5 or 6, most preferably in positions 4 or 6, in particular in position 4 of the anilide group [c.f. structure (I) above].
A stands preferably for the group A1.
A stands more preferably for A1 with the meaning 5-fluoro-1,3-dimethyl-1H-pyrazole-4-yl, 3-trifluormethyl-1-methyl-1H-pyrazole-4-yl or 3-difluoromethyl-1-methyl-1H-pyrazole-4-yl.
A stands most preferably for A1with the meaning 5-fluoro-1,3-dimethyl-1H-pyrazole-4-yl.
A stands moreover preferably for the group A2.
A stands more preferably for A2 with the meaning 2-trifluoromethylphenyl or 2-iodophenyl.
A stands moreover preferably for the group A3.
A stands more preferably for A3 with the meaning 1,4-dimethyl-pyrazole-3-yl, 1-methyl-4-trifluoromethyl-pyrazole-3-yl or 1-methyl-4-difluoromethyl-pyrazole-3-yl.
A stands most preferably for A3 with the meaning 1-methyl-4-trifluoromethyl-pyrazole-3-yl.
R² stands preferably for methyl or trifluoromethyl.
R³ stands preferably for hydrogen or fluorine.
R⁴ stands preferably for trifluoromethyl or iodine.
R⁵ stands preferably for trifluoromethyl.

The group definitions or explanations defined in general or within preferred ranges in the above can, however, also be combined arbitrarily with one another, that is between the respective ranges and preferred ranges. This applies to the end product as well as to the precursors and intermediates.

The definitions specified can also be combined with one another as desired. Moreover, individual definitions may be omitted.

Preferred, more preferred and most preferred are compounds of structure (I) which in each case bear the substituents defined as preferred, more preferred or most preferred.

Description of the Methods and Intermediate Products

Method (a)

If 1-methyl-4-trifluoromethyl)-1H-pyrrole-3-carbonyl chloride and {2-[(1S)-1,3,3-trimethylbutyl]-phenyl}amine are used as starting materials method (a) of the invention can be illustrated by the following reaction scheme:

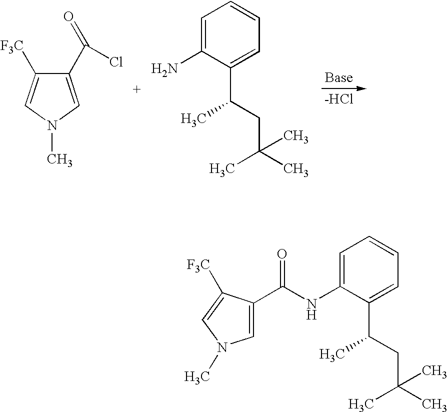

The carboxylic acid derivatives necessary as starting materials for the implementation of method (a) of the invention are defined in general terms by structure (II). In this structure (II) A has preferably, more preferably or most preferably those meanings which have been defined already as preferred, more preferred and most preferred for A in connection with the description of compounds of structure (I) of the invention. X¹ stands preferably for chlorine, bromine or hydroxy, more preferably for chlorine.

The carboxylic acid derivatives of structure (II) are known (c.f. WO 93/11117, EP-A 0 545 099, EP-A 0 589 301 and EP-A 0 589 313).

Furthermore, the amines necessary as starting materials for the implementation of method (a) of the invention are described in general terms by structure (III). In this structure (III) R and M have preferably, more preferably or most preferably those meanings which have been defined already as preferred, more preferred and most preferred for these groups in connection with the description of compounds of structure (I) of the invention.

The amines of structure (III) are new.

Amines of structure (III-a) 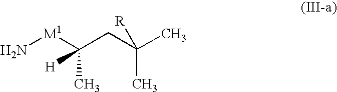
in which

R has the meanings defined above,

M¹ stands for M-1,

may be prepared for example in that

d) in a first step an aniline derivative of structure (VI) 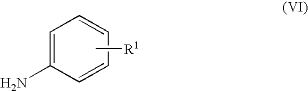
in which R¹ has the meanings defined above,
is reacted with an alkene of structure (VII) 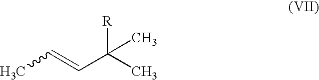
in which R has the meanings defined above,
in the presence of a catalyst, optionally in the presence of a base and optionally in the presence of a diluent,
and the alkenylaniline of structure (VIII) thus obtained 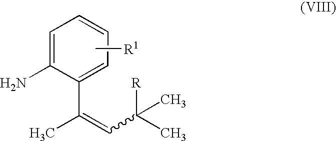
in which R and R¹ have the meanings defined above,
is hydrogenated in a second step optionally in the presence of a diluent and optionally in the presence of a catalyst,
and the racemic aniline derivative of structure (III-a-rac) thus obtained 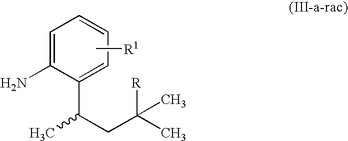
in which R and R¹ have the meanings defined above
is chromatographed in a third step on a chiral silica gel stationary phase in the presence of an eluent or eluent mixture as liquid phase.

The hydrogenation of compounds of structure (VIII) can also be carried out optionally in the presence of an optically active catalyst or in the presence of a catalyst and an optically active ligand and thus provide optically active compounds of structure (III-a).

Compounds of structure (III-a-rac) can also be fractionally crystallised in the presence of optically active acids under salt formation, following which the enantiomerically pure or enriched compounds of structure (III-a) is released. In general all optically active acids are suitable for the formation of diastereomeric salts. Examples are: (1S)-(+)camphor-10-sulphonic acid, (1R)-(−)-camphor-10-sulphonic acid, S,S-(−)-tartaric acid, R,R-(+)-tartaric acid, R-lactic acid, S-lactic acid or optically active amino acids, preferably naturally occurring optically active amino acids.

The aniline derivatives necessary as starting materials for the implementation of method (d) of the invention are defined in general terms by structure (VI). In this structure (VI) R¹ has preferably, more preferably or most preferably those meanings which have been defined already as preferred, more preferred and most preferred for these groups in connection with the description of compounds of structure (I) of the invention.

Aniline derivates of structure (VI) are known.

The alkenes necessary as starting materials for the implementation of method (d) of the invention are defined in general by structure (VII). In this structure (VII) R has preferably, more preferably or most preferably those meanings which have been described already as preferred, more preferred and most preferred for this group in connection with the description of compounds of structure (I) of the invention.

Alkenes of structure (VII) are known or can be obtained by known methods.

The alkenylaniline occurring as intermediates during implementation of method (d) of the invention are defined in general by structure (VIII). In this structure (VIII) R and R¹ have preferably, more preferably or most preferably those meanings which have been described already as preferred, more preferred and most preferred for these groups in connection with the description of compounds of structure (I) of the invention.

Alkenylanilines of structure (VIII) are known and/or can be obtained by known procedures.

The amines of structure (III-b) 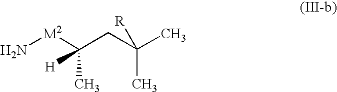
in which

R has the meanings defined above,

M² stands for M-2, M-3 or M-4,

may be obtained for example when

e) racemic amines of structure (III-b-rac) 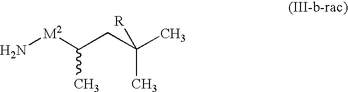
in which R and M² have the meanings defined above
are chromatographed on a chiral silica gel stationary phase in the presence of an eluent or eluent mixture as liquid phase.

The racemic amines of structure (III-b-rac) are known and/or can be obtained by known methods (c.f. e.g. WO 02/38542, EP-A 1 036 793 and EP-A 0 737 682).

Method (b)

The racemic compounds necessary as starting materials for the implementation of method (b) of the invention are defined in general by structure (I-rac). In this structure R, M and A stand preferably, more preferably or most preferably for those meanings which have been described already as preferred, more preferred and most preferred for these groups in connection with the description of compounds of structure (I) of the invention.

The racemic compounds of structure (I-rac) used in the implementation of method (b) are known and may be prepared by known methods (c.f. e.g. WO 03/010149, WO 02/38542 and DE-A 102 29 595). Racemic compounds of structure (I-rac) can be obtained, for example, by the reaction of carboxylic acid derivatives of structure (II) with racemic compounds of structures (III-a-rac) or (III-b-rac) in analogy to Method (a) of the invention.

In the implementation of Method (b) of the invention the methods of preparative chromatography are used, preferably the method of High Performance Liquid Chromatography (HPLC). Here a chiral silica gel stationary phase is used. Chiracel OD® has proved to be particularly suitable for the separation of compounds of structure (I-rac) into the two enantiomers. This separating material is commercially available. Other stationary phases may also be used as chromatographic material.

If compounds of structure (I-rac) are to be separated into the individual optically active compounds by fractional crystallisation all optically active acids are suitable for the formation of diastereomeric salts. Examples are: (1S)-(+)-camphor-10-sulphonic acid, (1R)-(−)-camphor-10-sulphonic acid, S,S-(−)-tartaric acid, R,R-(+)-tartaric acid, R-lactic acid, S-lactic acid or optically active amino acids, preferably naturally occurring optically active amino acids.

Method (c)

If N-[2-(1,3-Dimethylbut-1-en-1-yl)phenyl]-5-fluoro-1,3-dimethyl-1H-pyrazole-4-carboxamide, hydrogen and an optically active catalyst are used as starting materials Method (c) of the invention may illustrated by the following reaction scheme:

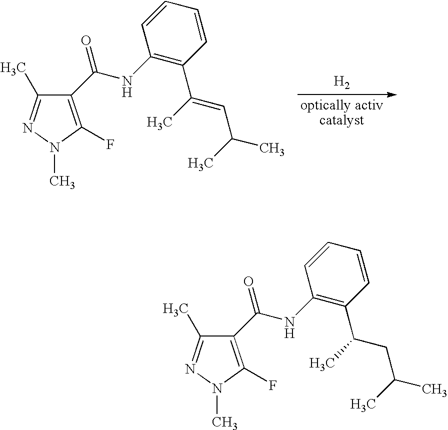

The compounds necessary as starting materials for the implementation of method (c) of the invention are defined in general by structures (IV) and (V). In these structures R, M and A have preferably, more preferably or most preferably those meanings which have been described already as preferred, more preferred and most preferred for these groups in connection with the description of compounds of structure (I) of the invention.

Compounds of structures (IV) and (V) (or mixtures of these compounds) are obtained when

f) carboxylic acid derivatives of structure (II) 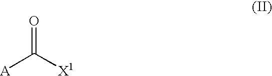
in which
A has the meanings defined above and
X¹ stands for halogen or hydroxy,
are reacted either with an alkenylaniline of structure (VIII) 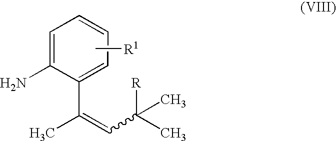
in which R and R¹ have the meanings defined above,
or with an alkenylaniline of structure (IX) 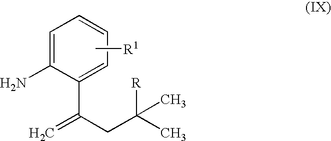
in which R and R¹ have the meanings defined above,
optionally in the presence of a catalyst, optionally in the presence of a condensation agent,
optionally in the presence of an acid binding agent and optionally in the presence of a diluent,
   or

g) carboxamides of structure (X) 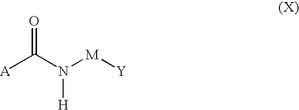
in which
M and A have the meanings defined above, and
Y stands for bromine or iodine,
are reacted with an alkene of structure (VII) 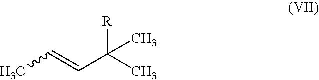
in which R has the meanings defined above,
or with an alkene of structure (XI) 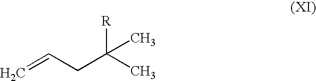
in which R has the meanings defined above,
in the presence of a catalyst, optionally in the presence of a base and optionally in the presence of a diluent.

The carboxylic acid derivatives of structure (II) necessary as starting materials in the implementation of method (f) of the invention have already been described in connection with method (a).

The alkenylanilines of structure (VIII) also necessary as starting materials in the implementation of method (f) of the invention have already been described in connection with method (d).

The alkenylanilines alternatively necessary as starting materials for the implementation of method (f) of the reaction are defined in general by structure (IX). In this structure (IX) R and R¹ have preferably, more preferably or most preferably those meanings which have been described already as preferred, more preferred and most preferred for these groups in connection with the description of compounds of structure (I) of the invention.

Alkenylanilines of structure (IX) are known and/or can be obtained by known methods.

The carboxamides necessary as starting materials for the implementation of method (g) of the reaction are defined in general by structure (X). In this structure (X) M and A have preferably, more preferably or most preferably those meanings which have been described already as preferred, more preferred and most preferred for these groups in connection with the description of compounds of structure (I) of the invention.

Carboxamides of structure (X) are known and/or can be obtained by known methods (c.f. WO 03/010149).

The alkenes of structure (VII) also necessary as starting materials for implementation of method (g) of the invention have already been described in connection with method (d).

The alkenes alternatively necessary as starting materials for the implementation of method (g) of the reaction are defined in general by structure (XI). In this structure (XI) R has preferably, more preferably or most preferably those meanings which have been described already as preferred, more preferred and most preferred for this group in connection with the description of compounds of structure (I) of the invention.

Alkenes of structure (XI) are known or can be obtained by known methods.

Reaction Conditions

All inert organic solvents are suitable as diluents for implementation of the methods (a) and (f) of the invention. These include preferably aliphatic, alicyclic or aromatic hydrocarbons such as, for example, petroleum ether, hexane, heptane, cyclohexane, methylcyclohexane, benzene, toluene, xylene or decalin; halogenated hydrocarbons such as, for example, chlorobenzene, dichlorobenzene, dichloromethane, chloroform, tetrachloromethane, dichloroethane or trichloroethane; ethers such as diethyl ether, diisopropyl ether, methyl-tert-butylether, methyl-tert-amyl ether, dioxan, tetrahydrofuran, 1,2-dimethoxyethane, 1,2-diethoxyethane or anisole, or amides such as N,N-dimethylformamide, N,N-dimethylacetamide, N-methylformanilide, N-methylpyrrolidone or hexamethylphosphor-amide.

Methods (a) and (f) of the invention are carried out optionally in the presence of a suitable acid acceptor. All normal inorganic or organic bases are suitable. These include preferably alkaline earth or alkali hydrides, hydroxides, amides, alkoxides, acetates, carbonates or -hydrogen carbonates such as, for example, sodium hydride, sodium amide, sodium methylate, sodium ethylate, potassium tert-butylate, sodium hydroxide, potassium hydroxide, ammonium hydroxide, sodium acetate, potassium acetate, calcium acetate, ammonium acetate, sodium carbonate, potassium carbonate, potassium hydrogen carbonate, sodium hydrogen carbonate or ammonium carbonate, as well as tertiary amines, such as trimethylamine, triethylamine, tributylamine, N,N-dimethylaniline, N,N-dimethyl-benzylamine, pyridine, N-methylpiperidine, N-methylmorpholine, N,N-dimethylaminopyridine, diazabicyclooctane (DABCO), diazabicyclononene (DBN) or diazabicycloundecene (DBU).

Methods (a) and (f) of the invention are optionally carried out in the presence of a suitable condensation agent. All condensation agents normally suitable for such amidation reactions can be used, for example acid halide formers such as phosgene, phosphorus tribromide, phosphorus trichloride, phosphorus pentachloride, phosphorus oxychloride or thionyl chloride; anhydride formers such as ethyl chloroformate, methyl chloroformate, isopropyl chloroformate, isobutyl chloroformate or methane sulphonyl chloride; carbodiimides such as N,N′-dicyclohexylcarbodiimide (DCC) or other standard condensation agents such as phosphorus pentoxide, polyphosphoric acid, N,N′-carbonyldi-imidazole, 2-ethoxy-N-ethoxycarbonyl-1,2dihydroquinoline (EEDQ), triphenyl phosphine/carbon tetrachloride or bromotripyrrolidinophosphonium hexafluorophosphate.

Methods (a) and (f) of the invention are optionally carried out in the presence of a catalyst, for example 4-dimethylaminopyridine, 1-hydroxybenzotriazole or dimethylformamide.

During the implementation of methods (a) and (f) of the invention the reaction temperature can be varied over a wide range. Normally temperatures of 0° C. to 150° C., preferably 0° C. to 80° C., are used.

For implementation of method (a) for the preparation of compounds of structure (I) 0.2 to 5 mol, preferably 0.5 to 2 mol, of the aniline derivative of structure (III) are normally used per mol of the carboxylic acid derivative of structure (II).

For implementation of method (f) for preparation of compounds of structures (IV) and (V) 0.2 to 5 mol, preferably 0.5 to 2 mol, of the alkenylaniline of structure (VIII) or (IX) are normally used per mol of the carboxylic acid derivative of structure (II).

All normal inert organic solvents, their mixtures, or their mixtures with water may be used as eluents in the implementation of method (b) of the invention. Preferably suitable are optionally halogenated aliphatic, alicyclic or aromatic hydrocarbons, such as petroleum ether, hexane, heptane, cyclohexane; dichloromethane, chloroform; alcohols such as methanol, ethanol, propanol; nitriles such as acetonitrile; esters such as methyl acetate or ethyl acetate. More preferable are aliphatic hydrocarbons such as hexane or heptane and alcohols such as methanol or propanol, most preferable are n-heptane and isopropanol or their mixtures.

During implementation of method (b) of the invention the reaction temperature can in each case be varied over a wide range. Normally temperatures between 10° C. und 60° C., preferably between 10° C. und 40° C., are used, more preferably room temperature.

During the implementation of Method (b) of the invention a ca. 1% solution of the racemic compound (I-rac) is used normally for chromatographic separation. However, it is also possible to use other concentrations. Work-up is carried out with normal procedures. The general procedure is that the eluate is highly concentrated, solid material is filtered off and dried after washing with n-heptane. The residue is optionally freed from impurities possibly still present by chromatography. Mixtures of n-hexane or cyclohexene or cyclohexene and ethyl acetate are used as eluents, the composition of which must be adjusted to the respective compound to be purified.

All inert organic solvents are suitable as diluent in the implementation of the first step of method (d) of the invention as well as method (g) of the invention. These include preferably nitriles such as acetonitrile, propionitrile, n- or i-butyronitrile or benzonitrile, or amides such as N,N-dimethylformamide, N,N-dimethylacetamide, N-methylformanilide, N-methylpyrrolidone or hexamethylphosphor-amide.

The first step of method (d) of the invention as well as method (g) of the invention are optionally carried out in the presence of a suitable acid acceptor. All normal inorganic and organic bases are suitable. These include preferably alkaline earth or alkali hydrides, hydroxides, amides, alkoxides, acetates, carbonates or hydrogen carbonates such as, for example, sodium hydride, sodium amide, sodium methylate, sodium ethylate, potassium tert-butylate, sodium hydroxide, potassium hydroxide, ammonium hydroxide, sodium acetate, potassium acetate, calcium acetate, ammonium acetate, sodium carbonate, potassium carbonate, potassium hydrogen carbonate, sodium hydrogen carbonate or ammonium carbonate, as well as tertiary amines, such as trimethylamine, triethylamine, tributyl-amine, N,N-dimethylaniline, N,N-dimethylbenzylamine, pyridine, N-methylpiperidine, N-methylmorpholine, N,N-dimethylaminopyridine, diazabicyclooctane (DABCO), diazabicyclononene (DBN) or diazabicycloundecene (DBU).

The first step of method (d) of the invention as well as method (g) of the invention are carried out in the presence of one or more catalysts. Particularly suitable are palladium salts or complexes. These include preferably palladium chloride, palladium acetate, tetrakis-triphenylphosphine)palladium or bis-triphenylphosphine)palladium dichloride. A palladium complex can also be produced in the reaction mixture when a palladium salt and a complex ligand are added separately to the reaction. Suitable ligands are preferably organophosphorus compounds, for example triphenylphosphine, tri-o-tolylphosphine, 2,2′-bis(diphenylphosphino)-1,1′-binaphthyl, dicyclohexylphosphinebiphenyl, 1,4-bis-(diphenylphosphino)butane, bisdiphenylphosphinoferrocene, di(tert-butylphosphino)biphenyl, di-(cyclohexylphosphino)biphenyl, 2-dicyclohexylphosphino-2′-N,N-dimethylaminobiphenyl, tricyclo-hexylphosphine, tri-tert-butylphosphine. The ligands may also be omitted.

The first step of method (d) of the invention as well as method (g) of the invention are also optionally carried out in the presence of a further metal salts such as copper salts, for example copper(I) iodide.

During the implementation of the first step of method (d) of the invention as well as method (g) of the invention the reaction temperatures may be varied over a wide range. Normally temperatures of 20° C. to 180° C., preferably temperatures of 50° C. bis 150° C., are used.

For implementation of the first step of method (d) of the invention for preparation of the alkenylanilines of structure (VIII) 1 to 5 mol, preferably 1 to 3 mol, of the alkene of structure (VII) are normally used per mol of the aniline derivative of structure (VI).

For implementation of method (g) for preparation of compounds of structures (IV) and (V) 1 to 5 mol, preferably 1 to 3 mol, of alkene of structure (VII) or (XI) are normally used per mol carboxamide of structure (X).

All inert organic solvents are suitable as diluent in the implementation of method (c) of the invention as well as the second step (hydrogenation) of method (d) of the invention. These include preferably aliphatic or alicyclic hydrocarbons such as, for example, petroleum ether, hexane, heptane, cyclohexane, methylcyclohexane or decalin; ethers such as diethyl ether, diisopropyl ether, methyl-tert-butyl ether, methyl-tert-amyl ether, dioxan, tetrahydrofuran, 1,2-dimethoxyethane or 1,2-diethoxyethane; alcohols such as methanol, ethanol, n- or iso-propanol, n-, iso-, sec- or tert-butanol, ethane-diol, propane-1,2-diol, ethoxyethanol, methoxyethanol, diethylene glycol monomethyl ether, diethylene glycol monoethyl ether, their mixtures with water or pure water.

The second step (hydrogenation) of method (d) of the invention is carried out in the presence of a catalyst. All catalysts usually used for hydrogenation are suitable. Examples are Raney nickel, palladium, ruthenium or platinum, optionally on a support such as, for example, active charcoal.

The chiral hydrogenation in the implementation of method (c) of the invention and in method (d) is carried out in the presence of an optically active ligand. Examples are the combination (R,R)-Me-DuPhos/RuCl₂® or (S,S)-Me-DuPhos/RuCl₂® (according to the desired enantiomer).

The hydrogenation in the second step of method (d) of the invention can also be carried out in the presence of triethylsilane instead of in the presence of hydrogen in combination with a catalyst.

During the implementation of method (c) of the invention as well as the second step of method (d) of the invention the reaction temperatures can be varied over a wide range. Normally temperatures of 0° C. to 150° C. are used, preferably at temperatures of 20° C. to 100° C.

Method (c) of the invention as well as the second step of method (d) of the invention are carried out under a hydrogen pressure between 0.5 and 200 bar, preferably between 2 and 50 bar, more preferably between 3 and 10 bar.

In each case, all normal inert organic solvents and their mixtures or possibly also mixtures with water are suitable for the implementation of the third step of method (d) of the invention and method (e) of the invention. Preferably suitable are optionally halogenated aliphatic, alicyclic or aromatic hydrocarbons, such as petroleum ether, hexane, heptane, cyclohexane; dichloromethane, chloroform; alcohols such as methanol, ethanol, propanol; nitriles such as acetonitrile; esters such as methyl acetate or ethyl acetate. More preferred are aliphatic hydrocarbons such as hexane or heptane and alcohols such as methanol or propanol, most preferred are n-heptane and isopropanol or their mixtures.

During implementation of the third step of method (d) of the invention and of method (e) of the invention the reaction temperatures can in each case be varied over wide range. In general temperatures between 10° C. and 60° C. are used, preferably between 10° C. und 40° C., more preferably at room temperature.

During the implementation of the third step of method (d) of the invention and method (e) a ca. 1% solution of the racemic compound (III-a-rac) and (III-b-rac), respectively, is normally used for chromatographic separation. However, it is also possible to use other concentrations. Work-up follows standard procedures. Normally the eluate is highly concentrated, solid material is filtered off and dried after washing with n-heptane. The residue is optionally freed from impurities possibly still present by chromatography. Mixtures of n-hexane or cyclohexane and ethyl acetate are used as eluents, the composition of which must be adjusted to the respective compound to be purified.

When not otherwise indicated all methods of the invention are normally carried out under normal pressure. It is also possible, however, to work under increased or reduced pressure—generally between 0.1 and 10 bar.

The compounds of the invention exhibit high microbicidal activity and can be used for the control of detrimental microorganisms such as fungi and bacteria in plant protection and material protection.

Fungicides may be used in plant protection for the control of Plasmodiophoromycetes, Oomycetes, Chytridiomycetes, Zygomycetes, Ascomycetes, Basidiomycetes and Deuteromycetes.

Bactericides may be used in plant protection for the control of Pseudomonadaceae, Rhizobiaceae, Enterobacteriaceae, Corynebacteriaceae and Streptomycetaceae.

By way of illustration, but not restricting, a number of pathogens of fungal and bacterial diseases which fall within the generic terms defined above is named:

Xanthomonas species such as, e.g. Xanthomonas campestris pv. oryzae;

Pseudomonas species such as, e.g. Pseudomonas syringae pv. lachrymans;

Erwinia species such as, e.g., Erwinia amylovora;

Pythium species such as, e.g., Pythium ultimum;

Phytophthora species such as, e.g., Phytophthora infestans;

Pseudoperonospora species such as, e.g., Pseudoperonospora humuli or Pseudoperonospora cubensis;

Plasmopara species such as, e.g., Plasmopara viticola;

Bremia species such as, e.g., Bremia lactucae;

Peronospora species such as, e.g., Peronospora pisi oder P. brassicae;

Erysiphe species such as, e.g., Erysiphe graminis;

Sphaerotheca species such as, e.g., Sphaerotheca fuliginea;

Podosphaera species such as, e.g., Podosphaera leucotricha;

Venturia species such as, e.g., Venturia inaequalis;

Pyrenophora species such as, e.g., Pyrenophora teres or P. graminea (conidial form: Drechslera, Syn: Helminthosporium);

Cochliobolus species such as, e.g., Cochliobolus sativus (conidial form: Drechslera, Syn: Helminthosporium);

Uromyces species such as, e.g., Uromyces appendiculatus;

Puccinia species such as, e.g., Puccinia recondita;

Sclerotinia species such as, e.g., Sclerotinia sclerotiorum;

Tilletia species such as, e.g., Tilletia caries;

Ustilago species such as, e.g., Ustilago nuda or Ustilago avenae;

Pellicularia species such as, e.g., Pellicularia sasakii;

Pyricularia species such as, e.g., Pyricularia oryzae;

Fusarium species such as, e.g., Fusarium culmorum;

Botrytis species such as, e.g., Botrytis cinerea;

Septoria species such as, e.g., Septoria nodorum;

Leptosphaeria species such as, e.g., Leptosphaeria nodorum;

Cercospora species, e.g., Cercospora canescens;

Alternaria species such as, e.g., Alternaria brassicae;

Pseudocercosporella species such as, e.g., Pseudocercosporella herpotrichoides,

Rhizoctonia species, such as, for example, Rhizoctonia solani.

The active compounds of the invention exhibit a high fortifying action in plants. They are thus suitable for the mobilisation of the plants' intrinsic resistance to infestation by detrimental microorganisms.

Within the present context plant fortifying (resistance inducing) compounds are understood to mean those compounds that are able to stimulate the defence mechanisms of plants such that the treated plants develop considerable resistance to detrimental microorganisms upon subsequent inoculation with these microorganisms.

In the present case detrimental microorganisms are understood to be phytopathogenic fungi, bacteria und viruses. The compounds of the invention can thus be used in order to protect plants against infestation by the named pathogens over a certain period of time. The time period within which protection is brought about ranges in general from 1 to 10 days, preferably 1 to 7 days after the treatment of the plants with the active compounds.

The good plant compatibility of the active compounds at the concentrations required for controlling plant diseases permits a treatment of above surface parts of the plants, plant and seed stock and the soil.

Thus the active compounds of the invention can be used with high success for the control of cereal diseases such as, for example, Puccinia species and diseases in wine, fruit and vegetable cultivation such as, for example, Botrytis, Venturia or Alternaria species.

The active compounds of the invention are also suitable to increase crop yields. They are moreover of low toxicity and exhibit a good plant compatibility.

At certain concentrations and applied quantities the active compounds of the invention can also be used as herbicides, for influencing plant growth and for the control of deadly pests. They can optionally also be used as intermediates and precursors for the synthesis of further active compounds.

According to the invention all plants and plant parts may be treated. By plants is meant all plants and plant populations such as desirable and undesirable wild plants or cultivated plants (including naturally occurring cultivated plants). Cultivated plants can be plants that can be obtained by conventional breeding and optimisation methods or by bioengineering or genetic engineering methods or by combinations of such methods, including transgenic plants and including plants varieties protected or not protected by plant varieties protection rights. By plant parts is meant all above ground and below ground parts and organs of the plants such as shoot, leaf, blossom and root, whereby as illustration leaves, needles, branches, trunks, blossoms, fruiting bodies, fruit and seed as well as roots, tubers, and rhizomes are listed. Harvested yields such as vegetative and generative propagation material, for example cuttings, tubers, rhizomes, shoots and seed also belong to plant parts.

The treatment according to the invention of plants and plant parts is carried out directly or by the action on their environment, habitat or storage facility with the normal treatment methods, e.g. by immersion, spraying, vaporising, misting, sprinkling, coating and with the propagation material, particularly with seeds, furthermore by single or multiple coating.

In material protection the compounds of the invention may be used for the protection of technical materials against infestation and destruction by detrimental microorganisms.

By technical materials is meant within the present context non-living materials which are produced for technical use. For example, technical materials that may be protected from microbial alteration or destruction by the compounds of the invention can be adhesives, glues, paper and cardboard, textiles, leather, wood, paint and plastic articles, cooling lubricants and other materials that can be attacked or destroyed my microorganisms. Within the concept of materials to be protected are also intended parts of production plants which may be impaired by the growth of microorganisms, such as cooling water cycles. Within the scope of the present invention technical materials mentioned are preferably adhesives, glues, paper and cardboard, leather, wood, paint, cooling lubricants and heat exchanger fluids are specially mentioned, more preferably wood.

Microorganisms which can effect a degeneration or an alteration in technical materials include for example, bacteria, fungi, yeasts, algae and moulds. The active compounds of the invention act preferably against fungi, especially mould fungi, wood discolouring and wood destroying fungi (Basidiomycetes) as well as moulds and algae.

Microorganisms of the following genuses are named as examples:

Alternaria, such as Alternaria tenuis,

Aspergillus, such as Aspergillus niger,

Chaetomium, such as Chaetomium globosum,

Coniophora, such as Coniophora puetana,

Lentinus, such as Lentinus tigrinus,

Penicillium, such as Penicillium glaucum,

Polyporus, such as Polyporus versicolor,

Aureobasidium, such as Aureobasidium pullulans,

Sclerophoma, such as Sclerophoma pityophila,

Trichoderma, such as Trichoderma viride,

Escherichia, such as Escherichia coli,

Pseudomonas, such as Pseudomonas aeruginosa,

Staphylococcus, such as Staphylococcus aureus.

Depending upon their respective physical and/or chemical properties the active compounds can be converted into the usual formulations such as solutions, emulsions, suspensions, powders, foams, pastes, granulates, aerosols, fine dispersion in polymeric materials and in coatings for seeds as well as cold and warm ULV spray formulations.

These formulations are prepared in the normal manner, e.g. by mixing the active compounds with diluents, that is liquid solvents, pressurised liquefied gases and/or solid supports, optionally with the use of surfactants, that is emulsifiers and/or dispersants and/or foaming agents. Where water is used as diluent organic solvents can also be used as cosolvents. Suitable liquid solvents essentially suitable are: aromatics such as xylene, toluene or alkylnaphthalenes, chlorinated aromatics or chlorinated aliphatic hydrocarbons such as chlorobenzenes, chloroethylenes or methylene chloride, aliphatic hydrocarbons, such as cyclohexane, or paraffins, for example petroleum fractions, alcohols such as butanol or glycol as well as their ethers and esters, ketones such as acetone, ethylmethylketone, isobutylmethylketone or cyclohexanone, highly polar solvents such as dimethylformamide and dimethyl sulphoxide, as well as water. By liquefied gaseous diluents or supports are meant such liquids that are gaseous at normal temperature and under normal pressure, for example, aerosol propellants such as halohydrocarbons as well as butane, propane, nitrogen and carbon dioxide. Suitable as solid supports are, e.g., natural mineral flours such as kaolin, argillaceous earth, talc, chalk, quartz, attapulgite montomorillonite or diatomaceous earth and synthetic mineral flours such as highly dispersed silica, aluminium oxide, and silicates. Suitable solid supports for granulates are, for example, broken and fractionated natural stone such as calcite, pumice, marble, sepiolite, dolomite as well as synthetic granulates from inorganic and organic flours as well as granulates from organic material such as sawdust, coconut shells, corn cobs and tobacco stems. Suitable emulsifiers and/or foaming agents are, e.g., non-ionic and anionic emulsifiers such as fatty acid esters of polyoxyethylene, fatty alcohol ethers of polyoxyethylene, for example, alkylaryl polyglycol ethers, alkyl sulphonates, alkyl sulphates, aryl sulponates and protein hydrolysates. Suitable dispersants are: e.g. lignin sulphite liquor and methyl cellulose.

Bonding agents such as carboxymethylcellulose, natural and synthetic powdery, granular or lactiferous polymers can be used in the formulation, such as gum Arabic, polyvinyl alcohol, polyvinyl acetate as well as natural phospholipids, such as cephalins and lecithins, and synthetic phospholipids. Further additives can be mineral and vegetable oils.

Colorants such as inorganic pigments, e.g. iron oxide, titanium oxide, ferrocyan blue and organic colorants such as alizarin, azo and metallophthalocyanin dyes and trace nutrients such as iron, manganese, boron, copper, cobalt, molybdenum and zinc salts can be used.

The formulations normally contain between 0.1 and 95% by weight active compound, preferably between 0.5 and 90%.

The active compounds of the invention can also be used as such or in their formulations in mixture with known fungicides, bactericides, miticides, nematocides or insecticides in order, for example, to broaden the spectrum of activity or to avoid the development of resistance. In many cases synergistic effects are obtained, that is the activity of the mixture is greater than the activity of the individual components.

For example, the following compounds are suitable as mixture partners:

Fungicides:

2-phenylphenol; 8-hydroxyquinoline sulphate; acibenzolar-S-methyl; aldimorph; amidoflumet; ampropylfos; ampropylfos-potassium; andoprim; anilazine; azaconazole; azoxystrobin; benalaxyl; benodanil; benomyl; benthiavalicarb-isopropyl; benzamacril; benzamacril-isobutyl; bilanafos; binapacryl; biphenyl; bitertanol; blasticidin-S; bromuconazole; bupirimate; buthiobate; butylamine; calcium polysulfide; capsimycin; captafol; captan; carbendazim; carboxin; carpropamid; carvone; chinomethionat; chlobenthiazone; chlorfenazole; chloroneb; chlorothalonil; chlozolinate; clozylacon; cyazofamid; cyflufenamid; cymoxanil; cyproconazole; cyprodinil; cyprofuram; dagger G; debacarb; dichlofluanid; dichlone; dichlorophen; diclocymet; diclomezine; dicloran; diethofencarb; difenoconazole; diflumetorim; dimethirimol; dimethomorph; dimoxystrobin; diniconazole; diniconazole-M; dinocap; diphenylamine; dipyrithione; ditalimfos; dithianon; dodine; drazoxolon; edifenphos; epoxiconazole; ethaboxam; ethirimol; etridiazole; famoxadone; fenamidone; fenapanil; fenarimol; fenbuconazole; fenfuram; fenhexamid; fenitropan; fenoxanil; fenpiclonil; fenpropidin; fenpropimorph; ferbam; fluazinam; flubenzimine; fludioxonil; flumetover; flumorph; fluoromide; flu-oxastrobin; fluquinconazole; flurprimidol; flusilazole; flusulfamide; flutolanil; flutriafol; folpet; fosetyl-A1; fosetyl-sodium; fuberidazole; furalaxyl; furametpyr; furcarbanil; furmecyclox; guazatine; hexachlorobenzene; hexaconazole; hymexazol; imazalil; imibenconazole; iminoctadine triacetate; iminoctadine tris(albesilate); iodocarb; ipconazole; iprobenfos; iprodione; iprovalicarb; irumamycin; isoprothiolane; isovaledione; kasugamycin; kresoxim-methyl; mancozeb; maneb; meferimzone; mepanipyrim; mepronil; metalaxyl; metalaxyl-M; metconazole; methasulfocarb; methfuroxam; metiram; metominostrobin; metsulfovax; mildiomycin; myclobutanil; myclozolin; natamycin; nicobifen; nitrothal-isopropyl; noviflumuron; nuarimol; ofurace; orysastrobin; oxadixyl; oxolinic acid; oxpoconazole; oxycarboxin; oxyfenthiin; paclobutrazol; pefurazoate; penconazole; pencycuron; phosdiphen; phthalide; picoxystrobin; piperalin; polyoxins; polyoxorim; probenazole; prochloraz; procymidone; propamocarb; propanosine-sodium; propiconazole; propineb; proquinazid; pro-thioconazole; pyraclostrobin; pyrazophos; pyrifenox; pyrimethanil; pyroquilon; pyroxyfur; pyrrolnitrine; quinconazole; quinoxyfen; quintozene; simeconazole; spiroxamine; sulphur; tebuconazole; tecloftalam; tecnazene; tetcyclacis; tetraconazole; thiabendazole; thicyofen; thifluzamide; thiophanate-methyl; thiram; tioxymid; tolclofos-methyl; tolylfluanid; triadimefon; tri-adimenol; triazbutil; triazoxide; tricyclamide; tricyclazole; tridemorph; trifloxystrobin; triflumizole; triforine; triticonazole; uniconazole; validamycin A; vinclozolin; zineb; ziram; zoxamide; (2S)-N-[2-[4-[[3-4-chlorophenyl)-2-propinyl]oxy]-3-methoxyphenyl]ethyl]-3-methyl-2-[(methylsulphonyl)-amino]-butanamide; 1-(1-naphthalenyl)-1H-pyrrole-2,5dione; 2,3,5,6tetrachloro-4-(methylsulphonyl)pyridine; 2-amino-4-methyl-N-phenyl-5-thiazole carboxamide; 2-Chloro-N-(2,3dihydro-1,1,3-trimethyl-1H-inden-4-yl)-3-pyridine carboxamide; 3,4,5-trichloro-2,6pyridine dicarbonitrile; Actino-vate; cis-1-(4chlorophenyl)-2-(1H-1,2,4-triazol-1-yl)-cycloheptanol; methyl 1-(2,3-dihydro-2,2-dimethyl-1H-inden-1-yl)-1H-imidazole-5-carboxylate; monopotassium carbonate; N-(6methoxy-3-pyridinyl)-cyclopropane carboxamide; N-butyl-8-(1,1-dimethylethyl)-1-oxaspiro[4.5]decane-3-amine; sodium tetrathiocarbonate; and copper salts and preparations such as Bordeaux mixture; copper hydroxide; copper naphthenate; copper oxychloride; copper sulphate; cufraneb; copper oxide; mancopper; oxine-copper.

Bactericides:

bronopol, dichlorophen, nitrapyrin, nickel dimethyldithiocarbamate, kasugamycin, octhilinon, furan carboxylic acid, oxytetracyclin, probenazol, streptomycin, tecloftalam, copper sulphate and other copper preparations.

Insecticides/miticides/nematocides:

1. Acetylcholinesterase (AChE) Inhibitors

1.1 Carbamates (e.g. alanycarb, aldicarb, aldoxycarb, allyxycarb, aminocarb, azamethiphos, bendiocarb, benfuracarb, bufencarb, butacarb, butocarboxim, butoxycarboxim, carbaryl, carbofuran, carbosulfan, chloethocarb, coumaphos, cyanofenphos, cyanophos, dimetilan, ethiofencarb, fenobucarb, fenothiocarb, formetanate, furathiocarb, isoprocarb, metam-sodium, methiocarb, methomyl, metolcarb, oxamyl, pirimicarb, promecarb, propoxur, thiodicarb, Thiofanox, triazamate, trimethacarb, XMC, xylylcarb)

1.2 Organophosphates (e.g. acephate, azamethiphos, azinphos(-methyl, -ethyl), bromophos-ethyl, bromfenvinfos(-methyl), butathiofos, cadusafos, carbophenothion, chlorethoxyfos, chlorfenvinphos, chlormephos, chlorpyrifos(-methyl/-ethyl), coumaphos, cyanofenphos, cyanophos, Chlorfenvinphos, demeton-S-methyl, demeton-S-methylsulphon, dialifos, diazinon, dichlofenthion, dichlorvos/DDVP, dicrotophos, dimethoate, dimethylvinphos, dioxabenzofos, disulfoton, EPN, ethion, ethoprophos, etrimfos, famphur, fenamiphos, fenitrothion, fensulfothion, fenthion, flupyrazofos, fonofos, formo-thion, fosmethilan, fosthiazate, heptenophos, iodofenphos, iprobenfos, isazofos, isofenphos, isopropyl O-salicylate, isoxathion, malathion, mecarbam, methacrifos, methamidophos, methidathion, mevinphos, monocrotophos, naled, omethoate, oxydemeton-methyl, parathion(-methyl/-ethyl), phenthoate, phorate, phosalone, phosmet, phosphamidon, phosphocarb, phoxim, pirimiphos(-methyl-ethyl), profenofos, propaphos, propetamphos, prothiofos, prothoate, pyraclofos, pyridaphenthion, pyridathion, quinalphos, sebufos, sulfotep, sulprofos, tebupirimfos, temephos, terbufos, tetrachlorvinphos, thiometon, triazophos, triclorfon, vamidothion)

2. Sodium Channel Modulators/Voltage Dependent Sodium Channel Blockers

2.1 Pyrethroides (e.g. acrinathrin, allethrin (d-cis-trans, d-trans), beta-cyfluthrin, bifenthrin, bioallethrin, bioallethrin S-cyclopentyl isomer, bioethanomethrin, biopermethrin, bioresmethrin, chlo-vaporthrin, cis-cypermethrin, cis-resmethrin, cis-permethrin, clocythrin, cycloprothrin, cyfluthrin, cyhalothrin, cypermethrin (alpha-, beta-, theta-, zeta-), cyphenothrin, DDT, deltamethrin, empenthrin (1R-isomer), esfenvalerate, etofenprox, fenfluthrin, fenpropathrin, fenpyrithrin, fenvalerate, flubrocythrinate, flucythrinate, flufenprox, flumethrin, fluvalinate, fubfenprox, gamma-cyhalothrin, imiprothrin, kadethrin, lambda-cyhalothrin, metofluthrin, permethrin (cis-, trans-), phenothrin (1R-trans isomer), prallethrin, profluthrin, protrifenbute, pyresmethin, resmethrin, RU 15525, silafluofen, tau-fluvalinate, tefluthrin, terallethrin, tetramethrin (1R-isomer), tralomethrin, transfluthrin, ZXI 8901, pyrethrins (pyrethrum))

2.2 Oxadiazines (e.g. indoxacarb)

3. Acetylcholine Receptor Agonists/Antagonists

3.1 Chloronicotinyles/neonicotinoides (e.g. acetamiprid, alothianidin, dinotefuran, imidacloprid, ni-tenpyram, nithiazine, thiacloprid, thiamethoxam)

3.2 Nicotine, bensultap, cartap

4. Acetylcholine Receptor Modulators

4.1 Spinosynes (e.g. spinosad)

5. GABA-Controlled Chloride Channel Antagonists

5.1 Cyclodiene organochlorines (e.g. camphechlor, chlordane, endosulfan, gamma-HCH, HCH, heptachlor, lindane, methoxychlor

5.2 Fiproles (e.g. Acetoprole, Ethiprole, Fipronil, Vaniliprole)

6. Chloride Channel Activators

6.1 Mectines (e.g. abamectin, avermectin, emamectin, emamectin benzoate, ivermectin, milbemectin, milbemycin)

7. Juvenile Hormone Mimetics

(e.g. diofenolan, epofenonane, fenoxycarb, hydroprene, kinoprene, methoprene, pyriproxifen, triprene)

8. Ecdysone Agonists/Disruptors

8.1 Diacylhydrazine (e.g. chromafenozide, halofenozide, methoxyfenozide, tebufenozide)

9. Chitin Biosynthesis Inhibitors

9.1 Benzoyl ureas (e.g. bistrifluron, chlofluazuron, diflubenzuron, fluazuron, flucycloxuron, flu-fenoxuron, hexaflumuron, lufenuron, novaluron, noviflumuron, penfluron, teflubenzuron, tri-flumuron)

9.2 Buprofezin

9.3 Cyromazine

10. Oxidative Phosphorylation Inhibitors, ATP Disruptors

10.1 Diafenthiuron

10.2 Organotins (e.g. azocyclotin, cyhexatin, fenbutatin oxide)

11. Decouplers of Oxidative Phosphorylation by Disruption of H-Proton Gradients

11.1 Pyrroles (e.g. chlorfenapyr)

11.2 Dinitrophenols (e.g. binapacyrl, dinobuton, dinocap, DNOC)

12. Site I Electron Transport Inhibitors

12.1 METI's (e.g. fenazaquin, fenpyroximate, pyrimidifen, pyridaben, tebufenpyrad, tolfenpyrad)

12.2 Hydramethylnone

12.3 Dicofol

13. Site II Electron Transport Inhibitors

13.1 Rotenones

14. Site III Electron Transport Inhibitors

14.1 Acequinocyl, fluacrypyrin

15. Microbial Insect Intestinal Membrane Disruptors

Bacillus thuringiensis strains

16. Fat Synthesis Inhibitors

16.1 Tetronic acids (e.g. spirodiclofen, spiromesifen)

16.2 Tetramic acids (e.g. 3-(2,5-dimethylphenyl)-8-methoxy-2-oxo-1-azaspiro[4.5]dec-3-en-4-yl ethyl carbonate (alias: carbonic acid, 3-(2,5-dimethylphenyl)-8-methoxy-2-oxo-1-azaspiro[4.5]dec-3-en-4-yl ethyl ester, CAS-Reg.-No.: 382608-10-8) and carbonic acid, cis-3-2,5-dimethylphenyl)-8-methoxy-2-oxo-1-azaspiro[4.5]dec-3-en-4-yl ethyl ester (CAS-Reg.-No.: 203313-25-1))

17. Carboxamides

(z.B. flonicamid)

18. Octopaminergic Agonists

(e.g. amitraz)

19. Magnesium-Stimulated ATPase Inhibitors

(z.B. propargite)

20. Phthalamides (e.g. N²-[1,1-Dimethyl-2-(methylsulfonyl)ethyl]-3-iodo-N¹-[2-methyl-4-[1,2,2,2-tetrafluoro-1-(trifluoromethyl)ethyl]phenyl]-1,2-benzene dicarboxamide (CAS-Reg.-No.: 272451-65-7))

21. Nereistoxin Analogues

(e.g. Thiocyclam hydrogen oxalate, thiosultap-sodium)

22. Biologics, Hormones or Pheromones

(e.g. azadirachtin, Bacillus spec., Beauveria spec., codlemone, Metarrhizium spec., Paecilomyces spec., Thuringiensin, Verticillium spec.)

23. Active Compounds with Unknown or Non-Specific Mechanisms of Action

23.1 Fumigation agents (e.g. aluminium phosphide, methyl bromide, sulfuryl fluoride)

23.2 Selective antifeedants (e.g. cryolite, flonicamide, pymetrozine)

23.3 Mite growth inhibitors (e.g. clofentezine, etoxazole, hexythiazox)

23.4 Aridoflumet, benclothiaz, benzoximate, bifenazate, bromopropylate, buprofezin, chinomethionat, chlordimeform, chlorobenzilate, chloropicrin, clothiazoben, cycloprene, dicyclanil, fenoxacrim, fentrifanil, flubenzimine, flufenerim, flutenzin, gossyplure, hydramethylnone, japonilure, metoxa-diazone, petroleum, piperonyl butoxide, potassium oleate, pyridalyl, sulfluramid, tetradifon, tetrasul, triarathene, verbutin

in addition the compound 3-methyl-phenyl-propyl carbamate (tsumacide Z), the compound 3-5-chloro-3-pyridinyl)-8-(2,2,2-trifluoroethyl)-8-azabicyclo[3.2.1]octane-3-carbonitrile (CAS-Reg.-Nr. 185982-80-3) and the corresponding 3-endo-isomer (CAS-Reg.-Nr. 185984-60-5) (c.f. WO-96/37494, WO-98/25923), and preparations which contain insecticidal plant extracts, nematodes, fungi or viruses.

A mixture with other known active compounds such as herbicides, or with fertilizers and growth regulators, safeners and semichemicals is also possible.

Moreover, the compounds of structure (I) of the invention exhibit very good antimycotic activity. They possess a very broad antimycotic spectrum of activity, especially against dermatophytes and blastomyces, mildew and diphasic fungi (e.g. against Candida species such as Candida albicans, Candida glabrata) and Epidermophyton floccosum, Aspergillus species such as Aspergillus niger and Aspergillus fumigatus, Trihophyton species such as Trichophyton mentagrophytes, Microsporon species such as Microsporon canis and audouinii. The listing of these fungi in no way represents a limitation of the recordable mycotic spectrum, it has only illustrative character.

The active compounds can be used as such, in the form of its formulations or the embodiments prepared from them, such as ready-for-use solutions, suspensions, wettable powders, pastes, soluble powders, dusts and granulates. Application is carried out in the normal manner, e.g. by pouring, spraying, nebulising, dusting, foaming, brushing, etc. It is further possible to apply the active compounds by the ultra low volume process or inject the active compound itself into the soil. It can also be used to treat the seeds of plants.

On using the active compounds as fungicides the amount applied can be varied over a large range according to the method of application. In the treatment of plant parts the amount of active compound applied lies generally between 0.1 and 10,000 g/ha, preferably between 10 and 1,000 g/ha. In the treatment of seed the amount of active compound applied lies generally between 0.001 and 50 g per kilogram seed, preferably between 0.01 and 10 g per kilogram seed. In the treatment of the soil the amount of active compound used lies usually between 0.1 and 10,000 g/ha, preferably between 1 and 5,000 g/ha.

As already described above, according to the invention all plants and their parts can be treated. In a preferred embodiment plant species and plant varieties occurring in the wild or obtained by conventional biological breeding methods such as crossing or protoplas fusion and their parts are treated. In a further preferred embodiment transgenic plants and plant varieties that were obtained by genetic engineering methods, possibly in combination with conventional methods (genetically modified organisms), and their parts are treated. The term “part” and “parts of plants” or “plant parts” were defined above.

Specially preferred according to the invention plants or the respective plant varieties available commercially or in use are treated. By plant varieties is meant plants with new properties (“traits”) that are bred both by conventional breeding, by mutagenesis or by recombinant DNA techniques. These can be varieties, strains, bio- or genotypes.

Depending upon the plant species or plant varieties, their position and conditions of growth (soil, climate, vegetation period, nutrition) superadditive (synergistic) effects can also occur by treatment according to the invention. Thus, for example, low application quantities and/or expansions of the spectrum of activity and/or an augmentation of the activity of the utilisable materials and agents of the invention, improved plant growth, increased tolerance towards high or low temperatures, increased tolerance to drought or to soil water or salt content, increased blossoming performance, easier harvesting, accelerated ripening, higher crop yields, improved quality and/or nutritional value of the harvested product, greater shelf-life, and/or processability of the harvested product are possible that go beyond the effects actually expected.

All plants which through genetic modification receive genetic material which impart these plants particularly advantageous properties (“traits”) belong to the preferred transgenic plants or plant varieties (obtained by genetic engineering) to be treated according to the invention. Examples of such properties are improved plant growth, increased tolerance to high and low temperatures, increased tolerance to drought and to soil water and salt content, increased blossoming performance, easier harvesting, accelerated ripening, higher crop yields, higher quality and/or nutritional value of the harvested product, longer shelf-life, and/or processability of the harvested product. Further and particularly highlighted examples of such properties are increased resistance of the plants to animal and microbial pests such as to insects, mites, pathogenic plant fungi, bacteria and/or viruses as well as an increased tolerance of the plants to certain active herbicidal compounds. As examples of transgenic plants are mentioned the important cultivated plants such as cereals (wheat, rice), maize, soya, potatoes, cotton, tobacco, rape as well as fruiting plants (with the fruits apples, pears, citrus fruits and grapes), whereby maize, soya, potatoes, cotton, tobacco and rape are specially mentioned. Particularly mentioned as properties (“traits”) are the increased resistance of the plants to insects, arachnids, nematodes, and slugs and snails through the toxins formed in the plants, especially those that are produced with genetic material from Bacillus Thuringiensis (e.g. with the genes CryIA(a), CryIA(b), CryIA(c), CryIIA, CryIIIA, CryIIIB2, Cry9c Cry2Ab, Cry3Bb and CryIF as well as their combinations) (hereinafter called “Bt plants”). Properties (“traits”) also particularly mentioned are the increased resistance of plants to fungi, bacteria and viruses through systemically acquired resistance (SAR), systemin, phytoalexine, elicitors and resistance genes and correspondingly expressed proteins and toxins. Further specially mentioned properties (“traits”) are the increased tolerance of the plants to certain active herbicidal compounds, e.g. imidazolines, sulphonyl ureas, glyphosates or phosphinotricin (e.g. “PAT” gene). The respective genes that impart the desired properties (“traits”) can also be present in combination in the transgenic plants. Examples of “Bt plants” are maize varieties, cotton varieties, soya varieties and potato varieties which are marketed under the brand names YIELD GARD® (e.g. maize, cotton, soya), KnockOut® (e.g. maize), StarLink® (e.g. maize), Bollgard® (cotton), Nucoton® (cotton) and NewLeaf® (potatoes). Mentioned as examples of herbicide tolerant plants are maize varieties, cotton varieties, and soya varieties which are marketed under the brand names Roundup Ready® (tolerance to glyphosates e.g. maize, cotton, soya), Liberty Link® (tolerance to phosphinotricin, e.g. rape), IMI® (tolerance to imidazolinones) and STS® (tolerance to sulphonyl ureas e.g. maize). Mentioned as examples of herbicide resistant plants (bred conventionally for herbicide tolerance) are varieties also marketed under the name Clearfield® (e.g. maize). Naturally these statements apply also to plant varieties which will be developed or marketed in the future with these genetic properties (“traits”) or those developed in the future.

According to the invention the plants described can be treated especially advantageously with the compounds of general structure (I) or the active compound mixtures of the invention. The preferred ranges described above for the active compounds or their mixtures apply also for the treatment of these plants. Particularly mentioned is plant treatment with the compounds or mixtures especially described in the present text.

The preparation and the use of the active compounds of the invention are described in the following examples.

### PREPARATION EXAMPLES

### Example 1

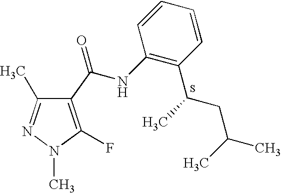

(±)-N-[2-1,3-Dimethylbutyl)phenyl]-5-fluoro-1,3-dimethyl-1H-pyrazole-4-carboxamide (200 mg) is dissolved in 25 ml n-heptane/isopropanol 9:1 (v/v=volume/volume). The solution is then fractionated by high performance liquid chromatography (HPLC) on the silica gel phase Chiralcel OD® [Manufacturer: Daicel (Japan), column dimensions: 500 mm×40 mm (i.d.), particle size: 20 μm, flow rate: 40 ml/min] with n-heptane/isopropanol 9:1 (v/v) as eluent. To separate the whole amount 5 ml proportions (each corresponding to 40 mg of the racemate) are applied to the column every 30 min. Detection of the compound is carried out with a UV detector at a wave length of 210 nm. After analytical investigation for enantiomeric purity the respective eluent fractions are combined and concentrated as far as possible in vacuum, the residues are filtered off and dried after washing with n-heptane. The crude product thus isolated is purified on silica gel (eluent: n-hexane/ethyl acetate 1:9→1:4, in each case v/v).

87 mg of N-{2-[(1S)-1,3-dimethylbutyl]phenyl}-5-fluoro-1,3-dimethyl-1H-pyrazole-4-carboxamide are obtained (melting point 52-54° C., rotation [α]_{D}=+6,7, c=0.87; methanol, 20° C., ee value=99%).

The enantiomeric purity of the carboxamides of structure (I) were determined by analytical HPLC under the following conditions:

| | |
|---|---|
| Separating phase: | Chiralcel OD ® (Daicel, Japan); 5 μm |
| Column: | 250 mm × 4.6 mm (I.D.) |
| Eluent: | n-heptane/2-propanol 10:1 |
| Flow rate: | 0.5 ml/min |
| UV detection: | 210 nm |

In a manner analogous to Example 1 and in accordance with the details in the general procedure description the compounds of structure (I) listed in the following table are obtained.

TABLE 1

| | |
|---|---|
| | (I) |
| 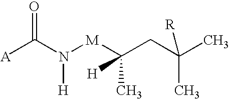 | |

| | | | | Log P | Rotation | ee |
|---|---|---|---|---|---|---|
| Ex. | R | M | A | (pH 2, 3) | [α]_{D} | value |
| 2 | CH₃ | 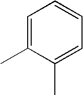 | 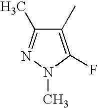 | 3.55 | −5.2 (c = 0.7; CHCl₃; 20° C.) | 99% |
| 3 | H | 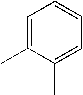 | 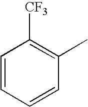 | 4.10 | −8.8 (c = 0.7; CHCl₃; 20° C.) | 99% |
| 4 | H | 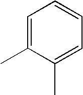 | 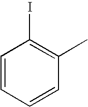 | 4.12 | −5.0 (c = 0.9; CHCl₃; 20° C.) | 97% |
| 5 | H | 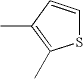 | 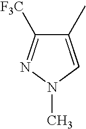 | 3.60 | +4.3 (c = 0.3; CH₃OH; 20° C.) | 95% |
| 6 | H | 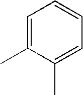 | 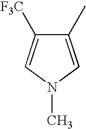 | 3.83 | −4.0 (c = 0.5; CH₃OH; 20° C.) | 99% |

The log P values given in the above table and in the preparation examples are determined according to EEC Directive 79/831 Annex V.A8 by HPLC (high performance liquid chromatography) on a reverse phase column (C 18) temperature: 43° C.

The determination is carried out in the acid region at pH 2.3 with 0.1% aqueous phosphoric acid and acetonitrile as eluent, linear gradient of 10% acetonitrile to 90% acetonitrile.

Calibration is carried out with non-branched alkane-2-ones (with 3 to 16 carbon atoms) whose log P value are known (determination of log P values by retention time by linear interpolation between two sequential alkanones).

The lambda max values were determined from UV spectra at 200 nm to 400 nm in the maxima of the chromatographic signals.

### APPLICATION EXAMPLES

### Example A

Podosphaera test (apple)/protective

| | | |
|---|---|---|
| Solvent: | 24.5 | parts by weight acetone |
| | 24.5 | parts by weight dimethylacetamide |
| Emulsifier: | 1 | part by weight alkylaryl polyglycol ether |

For the production of an appropriate active compound preparation 1 part by weight of active compound is mixed with the given amount of solvent and emulsifier and the concentrate is diluted to the desired concentration with water.

For the investigation for the protective activity young plants are sprayed with the active compound preparation in the amount specified. After drying of the spray coating the plants are inoculated with an aqueous spore suspension of the apple mildew pathogen Podosphaera leucotricha. The plants are then placed in a greenhouse at ca. 23° C. and a relative humidity of ca. 70%.

Evaluation is carried out 10 days after the inoculation. A level of activity of 0% corresponds to the level of activity of the control, whereas a level of activity of 100% means that no infestation is observed.

TABLE A
Podosphaera test (apple)/protective

| | Amount of | |
|---|---|---|
| | active | |
| | compound | Level of |
| Active | applied | activity |
| compound | in g/ha | in % |
| 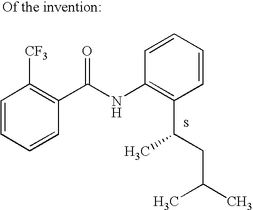 | 50 | 100 |
| 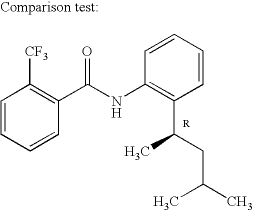 | 50 | 20 |
| 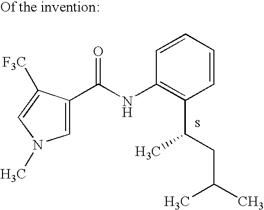 | 12.5 | 98 |
| 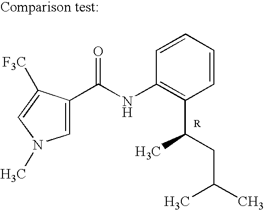 | 12.5 | 28 |

### Example B

Sphaerotheca test (cucumber)/protective

| | | |
|---|---|---|
| Solvent: | 24.5 | parts by weight acetone |
| | 24.5 | parts by weight dimethylacetamide |
| Emulsifier: | 1 | part by weight alkylaryl polyglycol ether |

For the production of an appropriate active compound preparation 1 part by weight of active compound is mixed with the given amount of solvent and emulsifier and the concentrate is diluted to the desired concentration with water.

For the investigation of the protective activity young cucumber plants are sprayed with the active compound preparation in the amount specified. After drying of the spray coating the plants are inoculated with an aqueous spore suspension of Sphaerotheca fuliginea. The plants are then placed in a greenhouse at ca. 23° C. and a relative humidity of ca. 70%.

Evaluation is carried out 7 days after the inoculation. A level of activity of 0% corresponds to the level of activity of the control, whereas a level of activity of 100% means that no infestation is observed.

TABLE B
Sphaerotheca test (cucumber)/protective

| | Amount of | |
|---|---|---|
| Active | active | |
| compound | compound | Level of |
| of the | applied | activity |
| invention | in g/ha | in % |
| 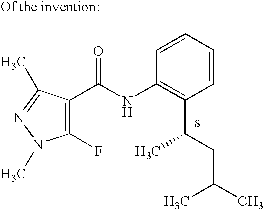 | 25 | 96 |
| 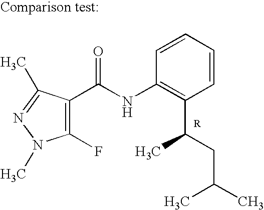 | 25 | 7 |
| 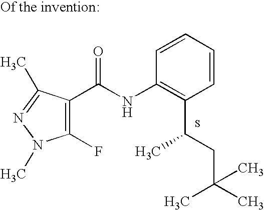 | 25 | 94 |
| 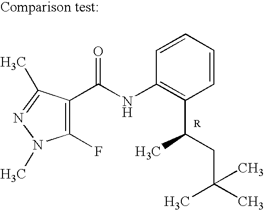 | 25 | 0 |
| 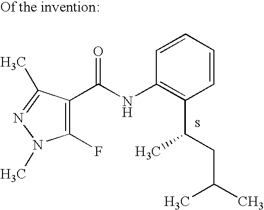 | 25 | 96 |
| 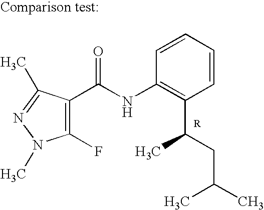 | 25 | 7 |
| 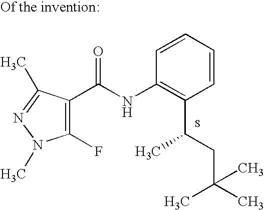 | 25 | 94 |
| 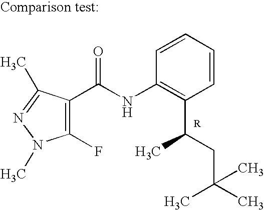 | 25 | 0 |
| 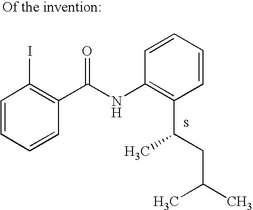 | 25 | 85 |
| 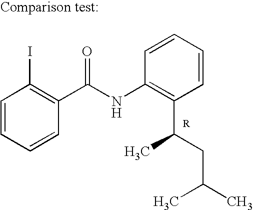 | 25 | 15 |
| 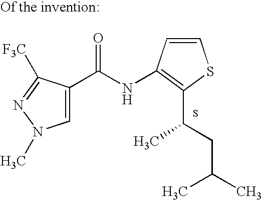 | 3.125 | 98 |
| 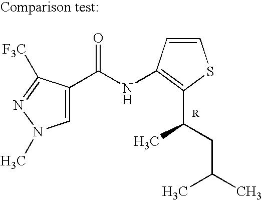 | 3.125 | 35 |
| 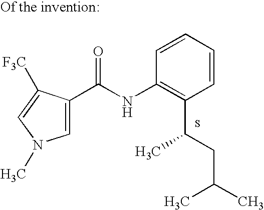 | 50 | 91 |
| 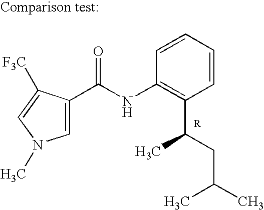 | 50 | 23 |

### Example C

Venturia test (apple)/protective

| | | |
|---|---|---|
| Solvent: | 24.5 | parts by weight acetone |
| | 24.5 | parts by weight dimethylacetamide |
| Emulsifier: | 1 | part by weight alkylaryl polyglycol ether |

For the production of an appropriate active compound preparation 1 part by weight of active compound is mixed with the given amount of solvent and emulsifier and the concentrate is diluted to the desired concentration with water.

For the investigation of the protective activity young plants are sprayed with the active compound preparation in the amount specified. After drying of the spray coating the plants are inoculated with an aqueous conidial suspension of the apple scab pathogen Venturia inaequalis then left for 1 day in an incubator at ca. 20° C. and a relative humidity of 100%.

The plants are then placed in a greenhouse at ca. 21° C. and a relative humidity of ca. 90%.

Evaluation is carried out 10 days after the inoculation. A level of activity of 0% corresponds to the level of activity of the control, whereas a level of activity of 100% means that no infestation is observed.

TABLE C
Venturia test (apple)/protective

| | Amount of | |
|---|---|---|
| Active | active | |
| compound | compound | Level of |
| of the | applied | activity |
| invention | in g/ha | in % |
| 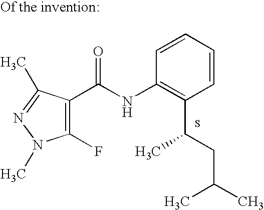 | 25 | 100 |
| 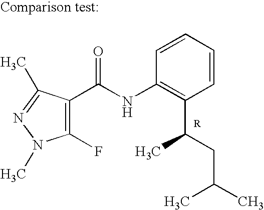 | 25 | 21 |
| 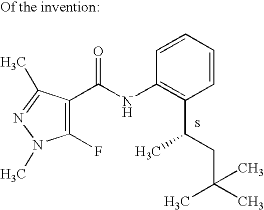 | 25 | 100 |
| 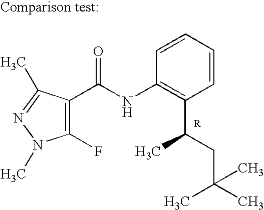 | 25 | 0 |
| 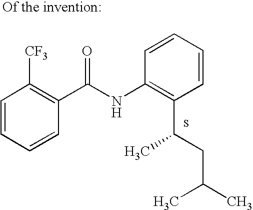 | 25 | 100 |
| 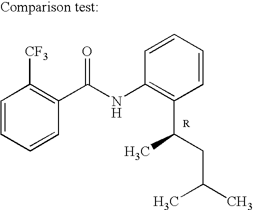 | 25 | 0 |
| 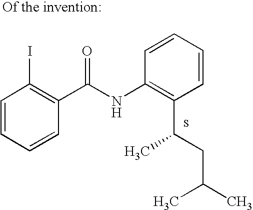 | 25 | 100 |
| 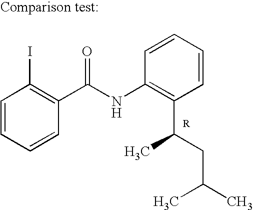 | 25 | 16 |
| 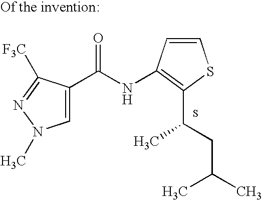 | 3.125 | 100 |
| 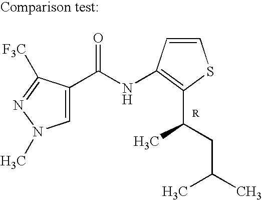 | 3.125 | 7 |
| 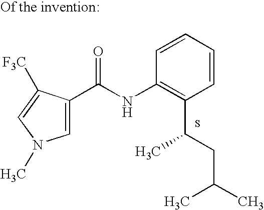 | 50 | 100 |
| 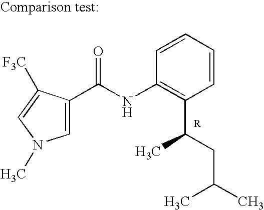 | 50 | 20 |

### Example D

Botrytis test (bean)/protective

| | | |
|---|---|---|
| Solvent: | 24.5 | parts by weight acetone |
| | 24.5 | parts by weight dimethylacetamide |
| Emulsifier: | 1 | part by weight alkylaryl polyglycol ether |

For the production of an appropriate active compound preparation 1 part by weight of active compound is mixed with the given amount of solvent and emulsifier and the concentrate is diluted to the desired concentration with water.

For the investigation the protective activity young plants are sprayed with the active compound preparation in the amount specified. After drying of the spray coating 2 small pieces of agar coated with Botrytis cinera are placed on each leaf. The inoculated plants are then placed in a darkened room at ca 20° C. and a relative humidity of 100%.

The size of the infestation spots on the leaves are evaluated 2 days after the inoculation. A level of activity of 0% corresponds to the level of activity of the control, whereas a level of activity of 100% means that no infestation is observed.

TABLE D
Botrytis test (bean)/protective

| | Amount of | |
|---|---|---|
| Active | active | |
| compound | compound | Level of |
| of the | applied | activity |
| invention | in g/ha | in % |
| 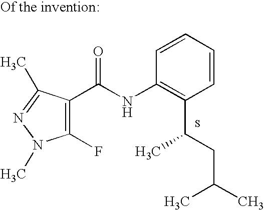 | 250 | 100 |
| 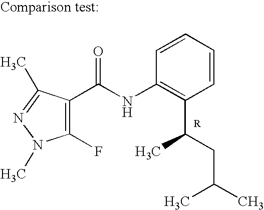 | 250 | 29 |
| 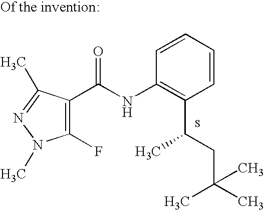 | 250 | 100 |
| 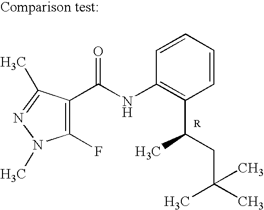 | 250 | 14 |
| 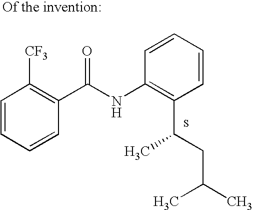 | 250 | 90 |
| 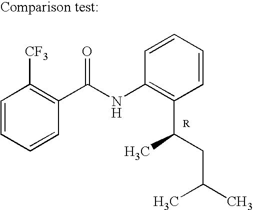 | 250 | 18 |
| 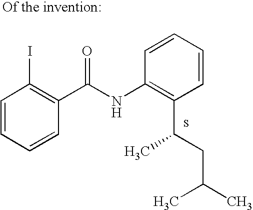 | 250 | 86 |
| 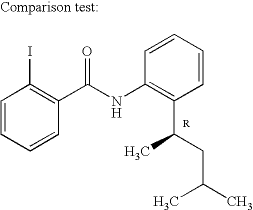 | 250 | 0 |
| 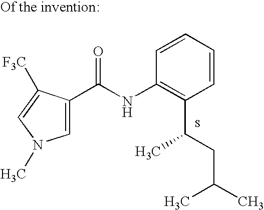 | 62.5 | 100 |
| 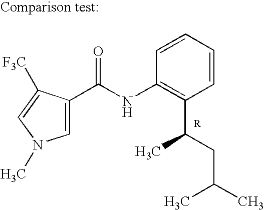 | 62.5 | 50 |

### Example E

Alternaria test (tomato)/protective

| | | |
|---|---|---|
| Solvent: | 24.5 | parts by weight acetone |
| | 24.5 | parts by weight dimethylacetamide |
| Emulsifier: | 1 | part by weight alkylaryl polyglycol ether |

For the production of an appropriate active compound preparation 1 part by weight of active compound is mixed with the given amount of solvent and emulsifier and the concentrate is diluted to the desired concentration with water.

For the investigation of the protective activity young plants are sprayed with the active compound preparation in the amount specified. After drying of the spray coating the plants are inoculated with an aqueous spore suspension of Alternaria solani. The plants are then placed in an incubator at ca. 20° C. and a relative humidity of 100%.

Evaluation is carried out 3 days after the inoculation. A level of activity of 0% corresponds the level of activity of the control, whereas a level of activity of 100% means that no infestation is observed.

TABLE E
Alternaria test (tomato)/protective

| | Amount of | |
|---|---|---|
| Active | active | |
| compound | compound | Level of |
| of the | applied | activity |
| invention | in g/ha | in % |
| 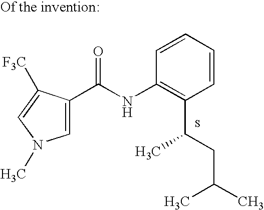 | 50 | 83 |
| 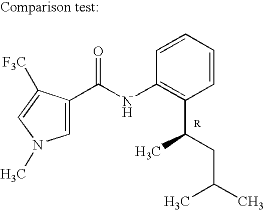 | 50 | 30 |

1. Optically active carboxamides of structure (I) 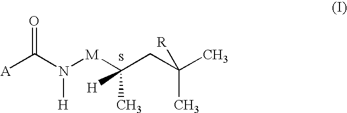
in which
R stands for hydrogen, fluorine, chlorine, methyl, ethyl or trifluoromethyl,
M stands for 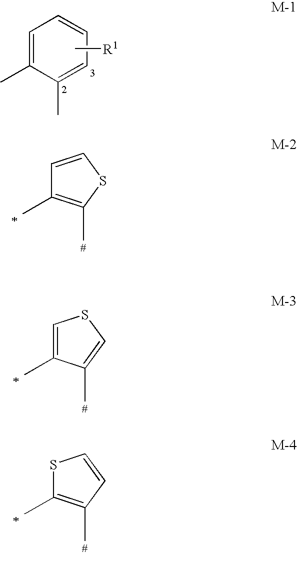
wherein the bond marked with * is coupled with the amide and the bond marked with # is coupled with the alkyl side chain,
R¹ stands for hydrogen, fluorine, chlorine, methyl or trifluoromethyl,
A stands for the group of structure (A1) 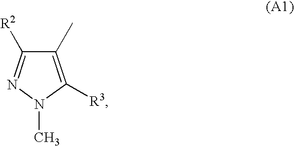
in which
R² stands for methyl, trifluoromethyl or difluoromethyl,
R³ stands for hydrogen, fluorine or chlorine,
or
A stands for the group of structure (A2) 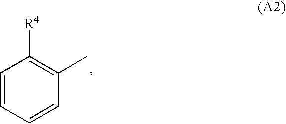
in which
R⁴ stands for trifluoromethyl, chlorine, bromine or iodine,
or
A stands for the group of structure (A3) 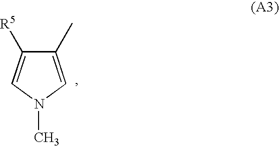
in which
R⁵ stands for methyl, trifluoromethyl or difluoromethyl.

2. Optically active carboxamides of structure (I) as claimed in claim 1, in which
R stands for hydrogen, methyl or ethyl,
M stands for M-1 or M-2,
R¹ stands for hydrogen, fluorine, chlorine, methyl or trifluoromethyl,
R² stands for methyl or trifluoromethyl,
R³ stands for hydrogen or fluorine,
R⁴ stands for trifluoromethyl or iodine,
R⁵ stands for trifluoromethyl.

3. Method for the preparation of optically active carboxamides of structure (I) as claimed in claim 1 characterised in that
a) carboxylic acid derivates of structure (II) 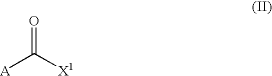
in which
A has the meanings defined in claim 1 and
X¹ stands for halogen or hydroxy,
is reacted with an amine of structure (III) 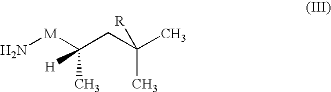
in which R and M have the meanings defined in claim 1,
optionally in the presence of a catalyst, optionally in the presence of a condensation agent, optionally in the presence of an acid binding agent and optionally in the presence of a diluent,
or
b) racemic compounds of structure (I-rac), 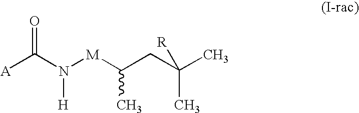
in which R, M and A have the meanings defined in claim 1,
are chromatographed on a chiral silica gel stationary phase in the presence of an eluent or an eluent mixture as the liquid phase,
or is fractionally crystallised with optically active acids under salt formation and subsequently the enantiomerically pure or enriched compounds of structure (I) is released,
or
c) compounds of structure (IV) 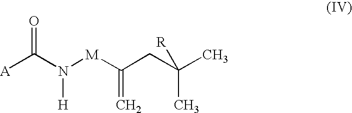
in which R, M and A have the meanings defined in claim 1,
or compounds of structure (V) 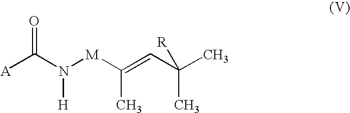
in which R, M and A have the meanings defined in claim 1,
or a mixture of both compounds is hydrogenated in the presence of an optically active catalyst or a catalyst with an optically active ligand.

4. Agent for the control of detrimental microorganisms characterised by a content of at least one optically active carboxamides of structure (I) as claimed in claim 1 together with diluents and/or surfactants.

5. Use of optically active carboxamides of structure (I) as claimed in claim 1 for the control of detrimental microorganisms.

6. Method for the control of detrimental microorganisms characterised in that optically active carboxamides of structure (I) as claimed in claim 1 are applied to the microorganisms and/or their habitat.

7. Method for the preparation of agents to control detrimental microorganisms characterised in that optically active carboxamides of structure (I) as claimed in claim 1 are mixed with diluents and/or surfactants.

8. Amines of structure (III) 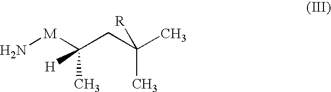
in which R and M have the meanings as defined in claim 1.
